(19) United States
(12) Patent Application Publication
(10) Pub. No.: US 2007/0123937 A1
(43) Pub. Date: May 31, 2007

(54) Apparatus for the emission of a combined flux of electrons and electromagnetic radiations particularly suitable for the treatment of the atheromatous disease

(76) Inventor: Giovanni Giuliani, Fenegro (IT)

Correspondence Address: BUCKNAM AND ARCHER, 1077 NORTHERN BOULEVARD, ROSLYN, NY, 11576, US

(21) Appl. No.: 10/573,844

(22) PCT Filed: Oct. 26, 2004
(86) PCT No.: PCT/EP04/12072

§ 371 Date: Mar. 29, 2006

(30) Foreign Application Priority Data
Nov. 6, 2003 (IT) MI2003A0002142

(51) Int. Cl. A61N 1/20

(52) U.S. Cl. 607/2

(57) ABSTRACT

An apparatus for the emission of a combined flux of electrons and electromagnetic radiations particularly suitable for the treatment of the atheromatous disease which includes: two electric circuits (2, 2′) with opposed polarity, each supplying a direct current with a voltage comprised between 4,000 and 80,000 V and an intensity comprised between 0.05 and 0.5 mA; two outputs (8, 8′); two plate terminal wires (9, 9′), each of them (9, 9′) being provided with at least one bundle of pointed wire elements (11, 11′), and means suitable for the identification of the coronary vessel corresponding to the stenosis or lesion to be treated and with control and drive means of the plate terminal wires so that the combined flux of electrons and electromagnetic radiations emitted is directed in a concentric and accurately targeted way towards the coronary vessel.

### FIELD OF THE INVENTION.

The present invention refers to an apparatus for the emission of a combined flux of electrons and electromagnetic radiations particularly suitable for the treatment of the atheromatous disease.

More particularly, the present invention refers to an apparatus for the emission of a combined flux of electrons and electromagnetic radiations particularly suitable for the treatment of the atheromatous disease wherein the stenosis or artery narrowing is not higher than 75%.

### DESCRIPTION OF PRIOR ART

As known, the atheromatous disease or atheromasia is the first cause of death in industrialized countries. The disease is due to a number of degenerative lesions or atheromas of arteries reducing the lumen of artery vessels. The atheroma, in the intimal wall of blood vessels, is constituted by a fatty degenerative plaque evolving into ulceration and subsequent covering of thrombi.

As known, the atheromatous plaque causes the ischaemia of the affected organ.

A consolidated and broad trial carried out on the atheromatous plaque in patients with atheromasia led to the definition of a stenosis limit within which atheromasia can be treated only with drugs.

The limit defined corresponds to 75%; therefore atheromasia with a stenosis which is not higher than 75% can be pharmaceutically treated; while the ones having a stenosis percentage higher than 75% are treated by invasive techniques such as angioplasty either with stent placement or not and surgical revascularization with by-pass.

For the purposes of the present invention, the stenosis percentage and the stenosis limit are determined by non-invasive methods such as the colour Doppler echocardiography and the TC angiography and by invasive methods such as the arteriography as described
1) ACC/AHA Guidelines for Percutaneous Coronary Intervention (Revision of the 1993 PTCA Guidelines);
2) ACC/AHA Guidelines for Coronary Artery Bypass Graft Surgery;
3) ACC/AHA 2002 Guideline Update for the Management of Patients With Chronic Stable Angina.

The pharmacological treatment, based on the use of many drugs, for atheromasia with a stenosis not higher than 75%, is not free from drawbacks even though it is currently used with satisfactory results.

One of these drawbacks is that the treatment effectiveness, in some cases, does not last; the treated plaques form again and reduce once again the blood flow (restenosis). Even though this drawback can be solved with the continuous use of drug, this involves a continuous dependence on the same drug.

Another drawback is due to the harmful action of these drugs, as regards the digestive tract, for the patient's health particularly if they are taken for long periods.

Moreover, the pharmacological treatment does not satisfactorily solve the atheromatous disease in all cases.

### DESCRIPTION OF THE INVENTION

Object of the present invention is to remove the above-mentioned drawbacks in the treatment of the atheromatous disease with a stenosis not higher than 75%

More particularly, object of the present invention is the effective prevention and/or treatment of the atheromatous disease with a stenosis not higher than 75% without using pharmacological treatments and in a non-invasive way.

A further object of the present invention is the provision of a means allowing the modulation of the stenosis in patients affected by atheromatous disease with values lower than 40% using techniques which are different from the pharmacological and invasive ones.

In its more general aspect, the present invention allows reaching these and other purposes resulting from the following description using a combined flux of electrons and electromagnetic radiations obtained supplying a direct current with a voltage comprised between 4,000 and 80,000 V and an intensity comprised between 0.05 and 0.5 mA to two bundles of pointed wire elements, one of which connected to a circuit with positive polarity (+) and the other one connected to a circuit with negative polarity (−) and directing said flux in a directed and targeted way towards the part of the body corresponding to the stenosis or lesion.

Said flux is directed and concentrated to the coronary vessel corresponding to the stenosis or lesion to be treated, the flux can be continuous, intermittent or a pulse flux; the intermittent flux is preferred as according to trials it proved to be the most efficacious in the treatment of the atheromatous disease.

Therefore, object of the present invention is, first of all, an apparatus for the emission of a combined flux of electrons and electromagnetic radiations comprising:
a. two electric circuits with opposed polarity, a circuit with positive polarity (+) and the other one with negative polarity (−) fed by the same alternate current distribution network (for example 220V), each circuit comprising an electric or electronic device transforming the alternate current of said distribution network into direct current and supplies a direct current with a voltage comprised between 4,000 and 80,000 V and an intensity comprised between 0.05 and 0.5 mA;
b. two outputs one of which is connected to the circuit with positive polarity (+) and the other one to the one with negative polarity (−);
c. two plate terminal wires, each of them being provided with at least one bundle of pointed wire elements, a wire being connected to the output of said circuit with positive polarity (+) and the other wire being connected to the output of said circuit with negative polarity (−);
d. means for the identification of the coronary vessel corresponding to the stenosis or lesion, and
e. control and drive means of said plate terminal wires in such a way that the combined flux of electrons and electromagnetic radiations emitted is directed in a concentric and accurately targeted way towards said coronary vessel.

Means for the identification of the coronary vessel to be treated can be constituted by a grid thoracic support wherein a mapping of the coronary tree obtained by a chest X-ray and a coronarography examination is configured. Said thoracic support is positioned and fixed to the patient body. For each single patient a mapping of the coronary tree is configured on the thoracic support by measure upon which vessels to be treated are reported together with the x-axis and the y-axis coordinates to set the goniometric measurements.

Control and drive means of the plate terminal wires can be traditional light means mounted on said plate terminal wires which are oriented and adjusted in such a way to light the area to be treated and have a visual control of the area involved with the treatment; or some LEDS o nano-emitters, at least three in number, which are positioned around the area to be treated and, if required, they are detected from special cameras positioned on the two plate terminal wires which control and adjust the position of said terminal wires.

Moreover, the apparatus for the emission of a combined flux of electrons and electromagnetic radiations object of the present invention can comprise a programmable timer placed on at least one of the two electric circuits for the emission of an intermittent flux at adjustable, programmed intervals.

An apparatus for the emission of a combined flux of electrons and electromagnetic radiations having the above-mentioned features from a) to c) is described in the U.S. Pat. No. 6,397,103 and is used in the treatment of decubitus ulcers, cicatrisation deficiency, skin and venous ulcers and sport traumas.

According to a second purpose, object of the present invention is the use of the above-described apparatus in the treatment and/or prevention of the atheromatous disease which consists in orienting the two bundles of pointed wire elements in a targeted and concentrated way towards the part of the body, insulated from ground, corresponding to the lesion to be treated and supplying between said bundles of pointed wires a direct current with a voltage comprised between 4,000 and 80,000 V and an intensity comprised between 0.05 and 0.5 mA.

For the treatment of the atheromatous disease with the apparatus of the present invention, the following operations are carried out:
an X-ray of the patient's chest is taken and by a coronarography examination, a mapping of the coronary tree by measure is configured on a thoracic support, upon it the vessels to be treated are reported;
the thoracic support is fixed on the patient's chest in such a way that the reported vessels to be treated correspond to the patient's ones;
the body corresponding to the lesion to be treated is insulated from the ground and exposed in correspondence of the two bundles of pointed terminal wires, a bundle of which is connected to the output of a direct current circuit with positive polarity (+) and the other bundle is connected to the output of a direct current circuit with a negative polarity (−);
the two bundles of pointed terminal wires are oriented in such a way that their fluxes are simultaneously directed in a targeted and concentrated way on the vessels to be treated;
the position and the orientation of said bundles of pointed wire elements is controlled and adjusted if required in such a way that the relevant fluxes are directed in a concentric and accurately targeted way towards the lesion to be treated, and
said part of the body is submitted to the action of a combined flux of electrons and electromagnetic radiations obtained supplying a direct current with a voltage comprised between 4,000 and 80,000 V and an intensity comprised between 0.05 and 0.5 mA between said electric circuits. Each bundle of pointed wire elements can have a cross section surface comprised between 0.1 and 100 mm², preferably between 1 and 10 mm² and it is formed by a number of wire elements comprised between 100 and 10,000. Each bundle can be divided into more groups of pointed wire elements and groups forming a bundle can be also 100.

The combined flux of electrons and electromagnetic radiations emitted by the apparatus is more significant and better distributed as higher is the number of wire elements forming each bundle.

The pointed wire elements are preferably carbon fibres. Any kind of carbon fibres can be used; said fibres are made of carbon for at least 90% by weight and they are generally obtained by carbonization of threadlike organic polymers, such as acrylic fibres.

### DESCRIPTION OF DRAWINGS

The apparatus for the emission of a combined flux of electrons and electromagnetic radiations of the present invention can be better understood from the following detailed description wherein reference is made to the Figures of the attached drawings representing an embodiment given only by way of illustrative and non-limitative example, wherein:

FIG. 1 is the electric scheme of this apparatus;

FIG. 2 is the front schematic view of this apparatus;

FIG. 3 is the front view of a bundle of carbon fibres obtained by a scanning electronic microscope with a ×24.3 magnification;

FIG. 4 is a perspective schematic view of a grid thoracic support for the orientation and centring in correspondence with the lesion to be treated, with the two bundles of pointed terminal wires of which one bundle is fed by direct current with positive polarity (+) and the other one with direct current with negative polarity (−), and

FIGS. 5 and 6 are perspective schematic views of articulated or flexible arms to support a plate terminal wire, respectively with and without said plate terminal wire.

### DESCRIPTION OF THE PREFERRED EMBODIMENT

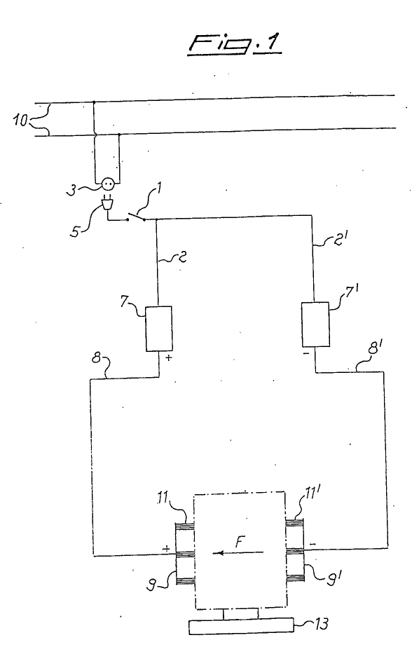

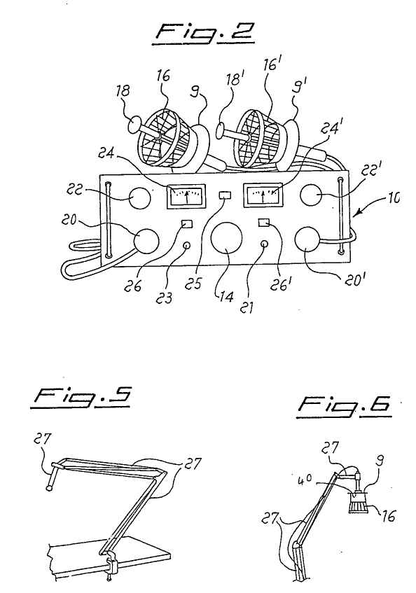

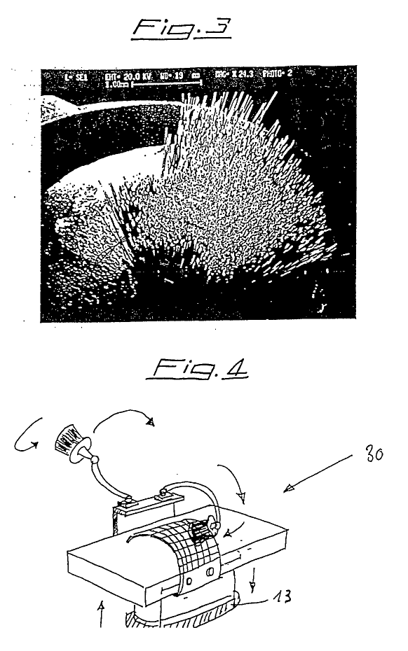

The apparatus for the emission of a combined flux of electrons and electromagnetic radiations of the present invention comprises an electric scheme shown in FIG. 1, comprising two electric circuits (2, 2′) fed by the same alternate current distribution network (10) (for example 220V a.c.) by an outlet (3), a plug (5) and a switch (1).

Each electric circuit (2, 2′) comprises an electric or electronic device (7, 7′) such as a transformer with rectifier diodes, which can feed each output circuit (8, 8′) with a direct current having an opposed polarity one from the other, a high voltage and a very low intensity.

In particular, said electric or electronic devices (7, 7′) supply the outputs (8, 8′) with direct currents with a voltage of 4,000-80,000 V and an intensity of 0.05-0.5 mA, one of which having a positive polarity (+) in a device (7) and the other one having a negative polarity (−) in the other device (7′).

The above-mentioned outputs (8, 8′) are respectively connected to two plate wires (9, 9′). Each plate wire (9, 9′) is provided with a bundle of carbon fibres (11, 11′), acting as pointed wire elements. Each bundle (11, 11′) can be divided into groups of carbon fibres, for example into three groups as shown in FIG. 1.

When the plate wires (9, 9′) and the relevant carbon fibres (11, 11′) are close to the part to be treated, the plug (5) is plugged into the outlet (3) and the switch (1) is closed, a flux of electrons and electromagnetic radiations circulating into the direction of the arrow F in FIG. 1 from the negative pole (−) to the positive one (+) is generated.

The flux of electrons and electromagnetic radiations can be continuous or intermittent; in case of intermittent flux the apparatus is provided with a programmable timer (14) opening the circuit at pre-defined intervals thus stopping the passage of current.

Plate wires (9, 9′) are made of conductive materials such as copper, aluminium, etc. and the bundle of carbon fibres (11, 11′) is fixed to each wire by any fastening means assuring the electrical connection between the plate wire (9, 9′) and each fibre of the bundle (11, 11′). Said plate wires (9, 9′) can have any shape, a square, rectangular, polygonal, circular, elliptic shape, etc. The surface of said wires (9, 9′) is not critical and depends from the surface of the injured part to be treated.

Each plate wire (9, 9′) with the bundle of carbon fibres (11, 11′) fixed to it can be protected by a removable casing (16, 16′) made of non-conductive insulating material. The removable casing (16, 16′) is preferably bell-shaped with the bundle of fibres fixed on the bottom part formed by the plate wire (9, 9′). The side wall of the casing (16, 16′) can be holed and provided with a cross support at the free end. A removable spacer (18, 18′) can be connected to said cross support, for example by a groove-and-tongue joint.

Both the casing (16, 16′) and the spacer (18, 18′) are preferably made of plastic material.

The casing (16, 16′) protects the carbon fibres from contact with the patient or the operator when the apparatus is used; the spacer (18, 18′) assures that the plate wire (9, 9′) or the carbon fibres are not in contact with the slin.

The plate wires (9, 9′) are connected to the apparatus by connection cables (20, 20′) with a plug.

Moreover, the apparatus can comprise a first output (21) for a cable with headset for the connection with the operator; a second output (23) for a cable to be connected to a removable headset for the connection with the patient and for each plate wire (9, 9′) a current regulator (22, 22′) and an amperemeter (24, 24′). Moreover, the apparatus can be provided with a pilot light (25) for control during operation, with power on buttons (26, 26′) of the two circuits and with a timer (14) for the emission of an intermittent flux.

Each plate wire (9, 9′) with the relevant casing (16, 16′) can be fixed to articulated or flexible arms (27) that make the orientation and the application to the various parts of the human body easy, placing the carbon fibres near the same body and not in contact with it; the spacers (18, 18′) assure that this contact is avoided.

For the positioning and the accurate orientation of the plate wires (9, 9′) and of the relevant pointed wire elements (11, 11′) towards the vessel to be treated, a thoracic support (30) which is fixed to the patient's body can be used. The two plate wires (9, 9′) are oriented and fixed on this thoracic support (30) with accurate measurements so that the flux of electrons and electromagnetic radiations generating among bundles (11, 11′) are directed towards the coronary vessel to be treated.

For each patient, starting from a chest X-ray and a coronarography examination, a mapping of the coronary tree is configured by measure on the thoracic support (30) upon which the vessels to be treated are reported together with the x-axis and the y-axis coordinates to set the goniometric measurements.

The patient is insulated from the ground by a dielectric element (13) which can be a chair, an armchair or a bed with insulating plastic material feet.

Before starting the treatment, a control must be carried out in order to assure that the combined flux of electrons and electromagnetic radiations is directed and concentrated on the part to be treated; for this purpose each plate (9, 9′) is provided with light means (40) and the arms (27) are adjusted and oriented in such a way that the area to be treated lights with the light beams emitted by said light means. In this way it is possible to visually control the area of the body to be treated.

The bundle of carbon fibres (11, 11′) fastened on each plate wire (9, 9′) can have a surface of the cross section comprised between 0.1 and 100 mm², preferably between 1 and 10 mm² and it is constituted by a number of carbon fibres comprised between 100 and 10,000. Each bundle can be divided into one or more groups of pointed wire elements and the groups forming a bundle can be also 100 in number.

The apparatus has been used to treat the atheromatous disease in patients with a stenosis or narrowing of arteries not higher than 75% determined by non-invasive diagnostic techniques such as the multilayer spiral computerized tomography with calcium score quantification.

According to the clinical medicine, the joining up of patients according to the following pathology classification was provided:
1. one-vessel, two-vessel coronary disease with a 60% stenosis in asymptomatic patient, no. 40 patients (males 38-females 2 average age 43 years old)
2. one-vessel, two-vessel coronary disease with a stenosis higher than 60% and lower than 75% in asymptomatic and symptomatic patient, No. 38 patients (males 37- females 1 average age 53 years old).

All patients were submitted to a 45 minute daily therapy at the maximum intensity allowed by the apparatus and a voltage of 40,000 V on the affected coronary vessel for 78 consecutive days, except on Sundays.

The following results were obtained:
stenosis characterized by soft plaques (up to 60%) that showed a hypoperfusion of the muscular tissue from a scintigraphy examination have all regressed after a negative final evaluation examination;
calcified stenosis comprised between 60 and 75% show a clear improvement of the blood flow particularly in cases of distal pathology of the vessel.

Even though the present invention has been described above with reference to one embodiment, it is understood that many variants and changes will be clear to people skilled in the art according to the above-mentioned description.

Therefore the present invention is meant to include all variants and changes falling within the spirit and the protective scope of the following claims.

1. An apparatus for the emission of a combined flux of electrons and electromagnetic radiations particularly suitable for the treatment of the atheromatous disease comprising:
a. two electric circuits (2, 2′) with opposed polarity, a circuit with positive polarity (+) and the other one with negative polarity (−) fed by the same alternate current distribution network (10), each circuit comprising an electric or electronic device (7, 7′) transforming the alternate current of said distribution network (10) into direct current and supplies a direct current with a voltage comprised between 4,000 and 80,0000 V and an intensity comprised between 0.05 and 0.5 mA;
b. two outputs (8, 8′) one of which (8) is connected to the circuit with positive polarity (+) and the other one (8′) to the one with negative polarity (−), and
c. two plate terminal wires (9, 9′), each of them (9, 9′) being provided with at least one bundle of pointed wire elements (11, 11′), a wire (9) being connected to the output of said circuit with positive polarity (+) and the other wire (9′) being connected to the output of said circuit with negative polarity (−), wherein said apparatus is provided with means suitable for the identification of the coronary vessel corresponding to the stenosis or lesion to be treated and with control and drive means of said plate terminal wires so that the combined flux of electrons and electromagnetic radiations emitted is directed in a concentric and accurately targeted way towards said coronary vessel.

2. The apparatus according to claim 1, wherein the means suitable for the identification of the coronary vessel to be treated are constituted by a thoracic support (30) wherein a mapping of the coronary tree obtained by a chest X-ray and a coronarography examination is configured and, by measure the vessels to be treated and the x-axis and y-axis coordinates to set the goniometric measurements.

3. The apparatus according to claim 1, wherein the command and drive means of the plate terminal wires are light means (40) mounted on said plate terminal wires which are oriented in such a way to light the area to be treated and have a visual control of the area involved with the treatment.

4. The apparatus according to claim 1, wherein the control and drive means of the plate terminal wires are LEDS or nano-emitters, at least three in number, which are positioned around the area to be treated and, if required, they are detected from special cameras positioned on the two plate terminal wires.

5. The apparatus according to claim 1, which comprises a programmable timer (14), placed on at least one of the two electric circuits for the emission of an intermittent flux or at adjustable intervals.

6. The apparatus according to claim 1, wherein the surface of the cross section of each bundle of pointed wire elements (11, 11′) is comprised between 0.1 and 100 mm².

7. The apparatus according to claim 6, wherein the surface of the cross section of each bundle of pointed elements (11, 11′) is comprised between 1 and 10 mm².

8. The apparatus according to claim 1, wherein each bundle (11, 11′) is constituted by a number of wire elements comprised between 100 and 10,000.

9. The apparatus according to claim 1, that wherein each bundle (11, 11′) is divided into many groups of pointed wire elements and the groups forming a bundle can be up to 100 in number.

10. The apparatus according to claim 1, wherein the pointed wire elements are carbon fibres.

11-12. (canceled)

13. The apparatus according to claim 1 for use in the treatment and/or prevention of the atheromatous disease, wherein the stenosis is not higher than 75%, consisting in orienting the two bundles of pointed wire elements (11, 11′) in a targeted and concentrated way towards the part of the body insulated from ground, corresponding to the stenosis or lesion to be treated, and delivering between said bundles (11, 11′) a direct current having a voltage comprised between 4,000 and 80,000 V and an intensity comprised between 0.05 and 0.5 mA.

14. The apparatus according to claim 13, wherein the delivery of the direct current is intermittent.
